(19) United States
(12) Patent Application Publication
(10) Pub. No.: US 2006/0249151 A1
(43) Pub. Date: Nov. 9, 2006

(54) Ventilator with rescuer and victim guidance

(75) Inventor: Anthony Gambone, Silver Springs, MD (US)

Correspondence Address: ROBERT D. FISH;RUTAN & TUCKER LLP, 611 ANTON BLVD 14TH FLOOR, COSTA MESA, CA, 92626-1931, US

(73) Assignee: China Resource Group, Inc.

(21) Appl. No.: 11/416,518

(22) Filed: May 2, 2006

Related U.S. Application Data
(60) Provisional application No. 60/677,473, filed on May 3, 2005.

(51) Int. Cl. A61M 16/00

(52) U.S. Cl. 128/204.18

(57) ABSTRACT

A breathing assistance device is provides guidance to the rescuer, guidance to the victim, and modifies treatment parameters automatically based upon feedback loops relating to patient needs. Guidance can include pictograms showing cartoons of lung expansion, airway patency, breathing rate, and depth of breathing. A microprocessor preferably produces the pictorial guidance as a function of at least one of estimated end tidal CO2, estimated fractional inspired oxygen, estimated fractional expired oxygen, estimated airway resistance, and estimated lung compliance. The microprocessor can also execute a software code that executes a feedback loop that attempts to normalize values of a parameter over time, by controlling at least one pressure in a neck pillow, mask pressure, breathing rate, breathing volume, inspiration time, and expiration time. Contemplated parameters can include estimated end tidal CO2, estimated fractional inspired oxygen, estimated fractional expired oxygen, estimated airway resistance, and estimated lung compliance.

This application claims priority to U.S. provisional application Ser. No. 60/677473 filed May 3, 2005.

### FIELD OF THE INVENTION

The field of the invention is emergency rescue equipment.

### BACKGROUND

Breathing is a complicated function, and providing assistance to a victim who is experiencing breathing difficulties is thought to require skilled intervention. A significant problem is that a great many situations require breathing assistance take place outside of a hospital or other health care facility in which sufficiently trained personal and equipment are available.

U.S. Pat. No. 6,289,890 to Bliss et al. (September 2001) addresses the skilled personnel problem by providing a portable rescue breathing assistance device that is said to provide semi-automated functionality. Devices contemplated in that patent provide verbal instructions to a rescuer, which nevertheless require some degree of training and expertise. For example, the rescuer must select victim size, and determine for himself whether the airway is patent. This and all other patents and referenced materials cited herein are incorporated by reference in their entirety.

Prior art devices also suffer from the lack of feedback loops. The '890 devices, for example, do not appear to have any feedback loop that would alter the rate, breathing depth or other treatment parameter other than merely switching between mandatory and spontaneous mode.

Still further, prior art devices suffer from a lack of guidance to the victim. Some emergency personnel, for example, would undoubtedly be better than others at calming a victim, and talking him through the situation. In addition, non-trained rescuers may well have little or no ability to do the above, or even to understand the situation.

What is still needed is a breathing assistance device that is more fully automated, preferably in terms of providing guidance to the rescuer, guidance to the victim, and modifying treatment parameters automatically based upon feedback loops relating to patient needs.

### SUMMARY OF THE INVENTION

The present invention provides systems and methods in which a breathing assistance device is more fully automated, preferably in terms of providing guidance to the rescuer, guidance to the victim, and modifying treatment parameters automatically based upon feedback loops relating to patient needs.

In preferred embodiments the pictogram shows a cartoon of lung, which can be a still image or some sort of animation. Other contemplated pictograms include representations of at least one of airway patency, breathing rate, and depth of breathing. Such pictograms can be displayed in any suitable manner, including for example a backlighted LCD and a plasma screen display. Especially preferred displays are pixel addressable.

In another aspect preferred ventilators comprise a microprocessor that produces the pictorial guidance as a function of at least one of estimated end tidal CO2, estimated fractional inspired oxygen, estimated fractional expired oxygen, estimated airway resistance, and estimated lung compliance. The microprocessor can also advantageously execute a software code that executes a feedback loop that attempts to normalize values of a parameter over time, by controlling at least one pressure in a neck pillow, mask pressure, breathing rate, breathing volume, inspiration time, and expiration time. Parameters, for example, can be selected from the list consisting of estimated end tidal CO2, estimated fractional inspired oxygen, estimated fractional expired oxygen, estimated airway resistance, and estimated lung compliance. In especially preferred embodiments the microprocessor can execute a software code that executes a pressure puff analysis that utilizes differences between successive estimated inspiration and expiration pressures, and can thereby estimate airway resistance and lung compliance.

Victim guidance can preferably comprise mindful breathing instructions and assurances.

### BRIEF DESCRIPTION OF THE DRAWING

FIG. 1 is a schematic of a breathing assist apparatus, showing a neck positioning device in a deflated configuration, and in functional positioning with respect to the head and neck of a victim.

FIG. 2 is a frame from an animation showing establishment of airway patency.

FIG. 3 is a series of bar graphs of showing airway patency 31, breathing rate 33, and depth of breathing 35.

FIG. 4 is a frame from an animation of lungs expanding and contracting

FIG. 5 is a graph depicting tidal CO₂ levels, and variances from an expected curve.

FIG. 6 is a graph of a graph of a pressure puff analysis.

### DETAILED DESCRIPTION

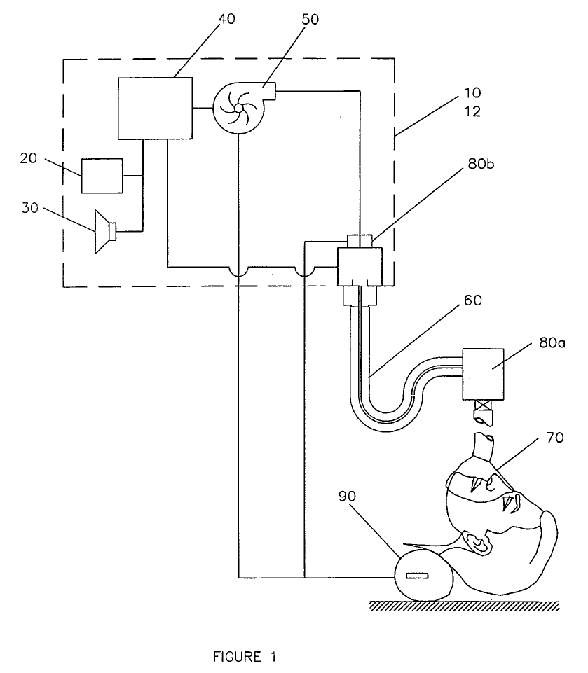

In FIG. 1 a ventilator 1 generally comprises a housing 10, a display 20, a speaker 30, a microprocessor 40, a source of pressurized gas 50, a tube 60, a mask 70, sensors 80a, 80b and a neck positioning device 90.

The housing 10 is preferably made of a durable polymer, and is as small as possible to house the various components. Housing 10 can advantageously include a carrying handle 12.

The display 20 is preferably a color LCD display, but can alternatively be a plasma screen or another display. Most preferably the display 20, as well as all the housing and all other components would meet any applicable military specifications. Display 20 can have any desired size and shape, but preferably measures at least 10 cm wide by 5 cm tall.

Speaker 30 is any suitable speaker providing sufficient loudness to instruct both a rescuer and a victim. A backup speaker (not shown) is also contemplated.

Microprocessor 40 can be any suitable off the shelf device, or a custom design, as long as it is adequate to run the contemplated software. A power supply 100 preferably provides power to all power consuming components of the ventilator 1. Power supply 100 is preferably rechargeable, and most preferably a rechargeable AC/DC supply. For reader understandability, the electrical connections among the power supply 100 and the electrical components are not shown.

The source of pressurized gas 50 is preferably a limited drag turbine flow generator, which uses ambient air, but is also contemplated to include pressurized oxygen (not shown). The source of pressurized gas 50 can also be used to inflate the neck positioning device 90.

The tube 60 and mask 70 can be standard devices, but more preferably comprise a co-axial breathing circuit with an integrated sensor. Sensors 80a, 80b can be disposed on, at or near the mask, or elsewhere in the ventilator as appropriate.

Neck positioning device 90 is described in concurrently filed provisional application “Neck Positioning Device For Mechanical Ventilator”, which is incorporated herein by reference in its entirety.

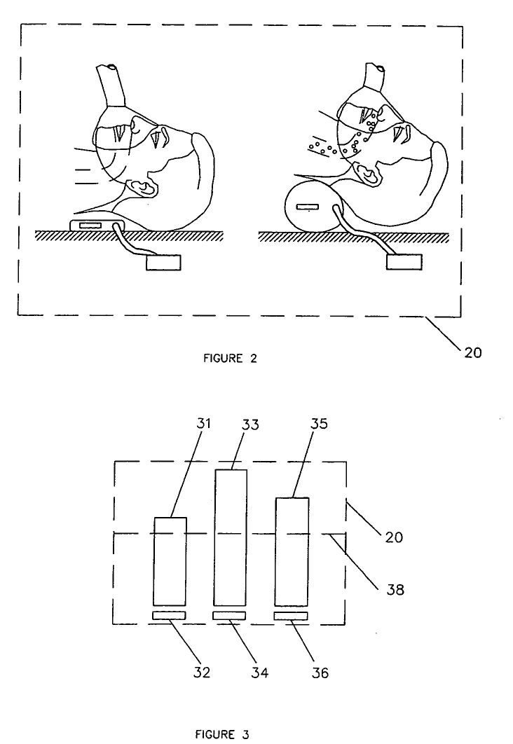

FIG. 2 shows the display 20 displaying an animation that shows establishment of airway patency.

FIG. 3 shows the display 30 displaying a series of bar graphs of airway patency 31, breathing rate 33, and depth of breathing 35. There are corresponding reference legends 32, 34, 36, and preferably a desired threshold marker 38.

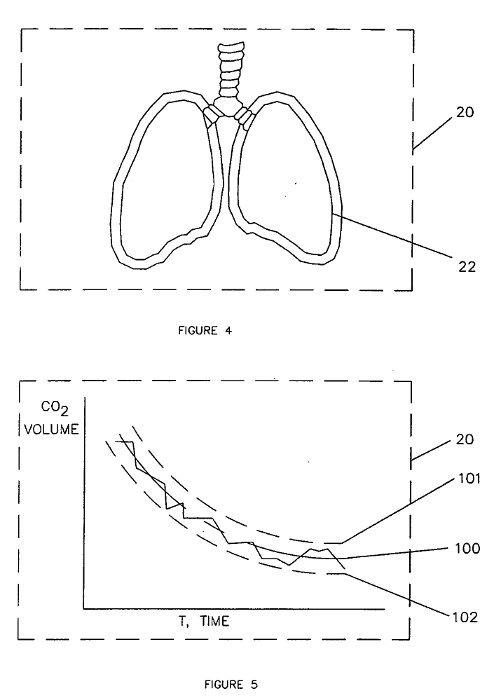

FIG. 4 shows the display 20 displaying an animation of lungs 22 expanding and contracting. In a contemplated embodiment the lungs 22 can change color depending upon the status of the victim's lungs.

The software produces the pictorial guidance as a function of at least one of estimated end tidal CO₂, estimated fractional inspired oxygen, estimated fractional expired oxygen, estimated airway resistance, and estimated lung compliance. Suitable equations can be derived from existing medical texts, including for example, “Automatic Weaning From Mechanical Ventilation Using An Adaptive Lung Ventilation Controller” Chest 106:6 (December 1994) pages 1843-1850; “Determination of Lung Volume in the ICU, H. Burchardi et al., Yearbook of Intensive Care and Emergency Medicine, Springer ISBN 3-540-63798-2.

The microprocessor can also advantageously execute a software code that executes a feedback loop that attempts to normalize values of a parameter over time, by controlling at least one pressure in a neck pillow, mask pressure, breathing rate, breathing volume, inspiration time, and expiration time. Normative values utilized in the software, for example, can be stored in a lookup table or represented by a series of lines of curves, which will likely vary over the duration of the treatment. Thus, a high end tidal CO₂ level would be expected to fall to a lower level during the course of treatment, whereas a low CO₂ would be expected to rise to a higher level during the course of treatment. This is depicted in FIG. 5, in which variances 101, 102 from an expected curve 100 are automatically corrected.

The feedback loop of the software may utilize variance from those expectations to alter breath rate, volume and so forth. Other parameters for which a feedback loop can be implements include estimated fractional inspired oxygen, estimated fractional expired oxygen, estimated airway resistance, and estimated lung compliance.

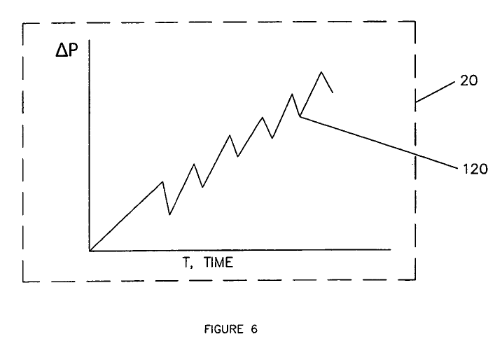

In especially preferred embodiments the microprocessor can execute a software code that executes a pressure puff analysis that utilizes differences between successive estimated inspiration and expiration pressures, and can thereby estimate airway resistance and lung compliance. FIG. 6 shows a graph of a pressure puff analysis. In this graph the data points of ΔP (inhalation pressure less exhalation pressure) are graphed against time to produce a curve 120. Area under the curve 120 relates to airway resistance and lung compliance.

It should be apparent to those skilled in the art that many more modifications besides those already described are possible without departing from the inventive concepts herein.

Moreover, in interpreting the disclosure, all terms should be interpreted in the broadest possible manner consistent with the context. In particular, the terms “comprises” and “comprising” should be interpreted as referring to elements, components, or steps in a non-exclusive manner, indicating that the referenced elements, components, or steps could be present, or utilized, or combined with other elements, components, or steps that are not expressly referenced.

What is claimed is:

1. A ventilator system that provides at least one of pictorial, musical, and voice guidance other than a voice prompt to a rescuer.

2. The ventilator system of claim 1 wherein the guidance comprises a pictogram of lung operation.

3. The ventilator system of claim 1 wherein the guidance comprises an animation of lung operation.

4. The ventilator system of claim 1 wherein the pictorial guidance comprises a representation of at least one of airway patency, breathing rate, and depth of breathing.

5. The ventilator system of claim 1 wherein the pictorial guidance comprises a representation of at least two of airway patency, breathing rate, and depth of breathing.

6. The ventilator system of claim 1 wherein the pictorial guidance comprises a representation of at least airway patency, breathing rate, and depth of breathing.

7. The ventilator system of claim 1 further comprising a microprocessor that produces the pictorial guidance as a function of at least one of estimated end tidal CO², estimated fractional inspired oxygen, estimated fractional expired oxygen, estimated airway resistance, and estimated lung compliance.

8. The ventilator system of claim 1 further comprising at least one of a backlighted LCD and a plasma screen display that displays the pictorial guidance.

9. The ventilator system of claim 1 further comprising at pixel addressable display that displays the pictorial guidance.

10. The ventilator system of claim 1 further comprising a microprocessor that executes a software code that executes a feedback loop that attempts to normalize values of a parameter over time, by controlling at least one pressure in a neck pillow, mask pressure, breathing rate, breathing volume, inspiration time, and expiration time.

11. The ventilator system of claim 10 wherein the parameter is selected from the list consisting of estimated end tidal CO², estimated fractional inspired oxygen, estimated fractional expired oxygen, estimated airway resistance, and estimated lung compliance.

12. The ventilator system of claim 1 further comprising a microprocessor that executes a software code that executes a pressure puff analysis that utilizes differences between successive estimated inspiration and expiration pressures.

13. The ventilator system of claim 10 wherein the microprocessor further executes the software to estimate airway resistance and lung compliance.

14. The ventilator system of claim 1 wherein the guidance comprises voice guidance to a victim.

15. The ventilator system of claim 14 wherein the voice guidance comprises mindful breathing instructions.

16. A ventilator system that provides a pictorial guidance to a rescuer and voice guidance to a victim.

17. The ventilator system of claim 16 wherein the pictorial guidance comprises a representation of at least one of airway patency, breathing rate, and depth of breathing.

18. The ventilator system of claim 16 further comprising a microprocessor that produces the pictorial guidance as a function of at least one of estimated end tidal CO², estimated fractional inspired oxygen, estimated fractional expired oxygen, estimated airway resistance, and estimated lung compliance.

19. The ventilator system of claim 16 wherein the pictorial guidance comprises a representation of at least one of airway patency, breathing rate, and depth of breathing, and the voice guidance comprises mindful breathing instructions.
